SEQUENCE LISTING (14 sequences)

SEQ ID NO: 1 - DNA, 449 bp, RAT
cccaaaccggacaccatgaagatcccaattcttcccatcgtggctctcctctctcttctggcattgcatgcagcccagggagcagccctggggacgcctatggaagacaccacctccagtaattacccttcgggaacagagggccttagtgagttcctgaactttaacaagttacagtctgcctttaagtcggatgacttcctgaactggcacgtcctcactgatatgttcaaaaaagcgcttcctttcattaactgggaattcttccctaaggtgaaaggactgagaagcgcagttcctgattcccagtgaccattctctttaagacctaggacctaggctgaccccactagaagaggagccaagtgtggaatccgaagtccatcctacgacacatctcccctggctcagtacctccaataaagtgttttccactcgaaaaaaaaaaa

SEQ ID NO: 2 - protein, 98 aa, RAT
MKIPILPIVALLSLLALHAAQGAALGTPMEDTTSSNYPSGTEGLSEFLNFNKLQSAFKSDDFLNWHVLTDMFKKALPFINWEFFPKVKGLRSAVPDSQ

SEQ ID NO: 3 - DNA, 447 bp, Homo sapiens
gaattcggcacgaggaaacttgacgccatgaagatcccggtccttcctgccgtggtgctcctctccctcctggtgctccactctgcccagggagccaccctgggtggtcctgaggaagaaagcaccattgagaattatgcgtcacgacccgaggcctttaacaccccgttcctgaacatcgacaaattgcgatctgcgtttaaggctgatgagttcctgaactggcacgccctctttgagtctatcaaaaggaaacttcctttcctcaactgggatgcctttcctaagctgaaaggactgaggagcgcaactcctgatgcccagtgaccatgacctccactggaagagggggctagcgtgagcgctgattctcaacctaccataactctttcctgcctcaggaactccaataaaacattttccatccaaaaaaaaaaaaaaaaaaaa

SEQ ID NO: 4 - protein, 99 aa, Homo sapiens
MKIPVLPAVVLLSLLVLHSAQGATLGGPEEESTIENYASRPEAFNTPFLNIDKLRSAFKADEFLNWHALFESIKRKLPFLNWDAFPKLKGLRSATPDAQ

SEQ ID NO: 5 - DNA, 379 bp, Homo sapiens
ggcacgaggaaacttgacgccatgaagatcccggtccttcctgccgtggtgctcctctccctcctggtgctccactctgcccagggagccaccctgggtggtcctgaggaagaaagcaccattgagaattatgcgtcacgacccgaggcgtttaaggctgatgagttcctgaactggcacgccctctttgagtctatcaaaaggaaacttcctttcctcaactgggatgcctttcctaagctgaaaggactgaggagcgcaactcctgatgcccagtgaccatgacctccactggaagagggggctagcgtgagcgctgattctcaacctaccataactctttcctgcctcaggaactccaataaaacattttccatcc

SEQ ID NO: 6 - protein, 85 aa, Homo sapiens
MKIPVLPAVVLLSLLVLHSAQGATLGGPEEESTIENYASRPEAFKADEFLNWHALFESIKRKLPFLNWDAFPKLKGLRSATPDAQ

SEQ ID NO: 7 - DNA, 454 bp, Mus musculus
gaattcggcttcagcccaaaccggacaccatgaagatcccaattcttcccgtcgtggctctcctctctcttctggcattgcatgcggtccagggagcagccctggggcatcccacgatatacccggaagatagcagctacaataattaccctaccgcaacagagggccttaacaatgagttcctgaactttaagaggctacagtctgcctttcagtcagaaaacttcctgaactggcacgtcatcactgatatgttcaaaaatgcatttcctttcattaactgggacttcttccctaaggtgaaaggactgagaagtgccgctcctgattcccagtgaccattctctttaagacctaggacctaggctgagcccagtagaagaggaggcaagcatggaatctgaagtccatcctacgacaaatcttccttgcctcagttccccaagccgaattc

SEQ ID NO: 8 - protein, 102 aa, Mus musculus
MKIPILPVVALLSLLALHAVQGAALGHPTIYPEDSSYNNYPTATEGLNNEFLNFKRLQSAFQSENFLNWHVITDMFKNAFPFINWDFFPKVKGLRSAAPDSQ

SEQ ID NO: 9 - DNA, 445 bp, Mus musculus
gaattcggcttcagcccaaaccggacaccatgaagatcccaattcttcccgtcgtggctctcctctctcttctggcattgcatgcggtccagggagcagccctggggcatcccacggaagatagcagctacaataattaccctaccgcaacagagggccttaacaatgagttcctgaactttaagaggctacagtctgcctttcagtcagaaaacttcctgaactggcacgtcatcactgatatgttcaaaaatgcatttcctttcattaactgggacttcttccctaaggtgaaaggactgagaagtgccgctcctgattcccagtgaccattctctttaagacctaggacctaggctgagcccagtagaagaggaggcaagcatggaatctgaagtccatcctacgacaaatcttccttgcctcagttccccaagccgaattc

SEQ ID NO: 10 - protein, 99 aa, Mus musculus
MKIPILPVVALLSLLALHAVQGAALGHPTEDSSYNNYPTATEGLNNEFLNFKRLQSAFQSENFLNWHVITDMFKNAFPFINWDFFPKVKGLRSAAPDSQ

SEQ ID NO: 11 - DNA, 359 bp, Mus musculus
gaattcggcttcagcccaaaccggacaccatgaagatcccaattcttcccgtcgtggctctcctctctcttctggcattgcatgcggtccagggagcagccccggggcatcccacgatatacccggaagatagcagctacaataattaccctaccgcaacagaggcctttcagtcagaaaacttcctgaactggcacgtcatcactgatatgttcaaaaatgcatttcctttcattaactgggacttcttccctaagacctaggacctaggctgagcccagtagaagaggaggcaagcatggaatctgaagtccatcctacgacaaatcttccttgcctcagttccccaagccgaattc

SEQ ID NO: 12 - protein, 77 aa, Mus musculus
MKIPILPVVALLSLLALHAVQGAAPGHPTIYPEDSSYNNYPTATEAFQSENFLNWHVITDMFKNAFPFINWDFFPKT

SEQ ID NO: 13 - DNA, 401 bp, Mus musculus
gaattcggcttcagcccaaaccggacaccatgaagatcccaattcttcccgtcgtggctctcctctctcttctggcattgcatgcggtccagggagcagccctggggcatcccacgatatacccggaagatagcagctacaataattaccctaccgcaacagagggccttaacaatgagttcctgaactttaagaggctacagtctgcctttcagtcagaaaacttcctgaactggcacgtcatcactgatatgttcaaaaatgcatttcctttcattaactgggacttcttccctaagacctaggacctaggctgagcccagtagaagaggaggcaagcatggaatctgaagtccatcctacgacaaatcttccttgcctcagttccccaagccgaattc

SEQ ID NO: 14 - protein, 91 aa, Mus musculus
MKIPILPVVALLSLLALHAVQGAALGHPTIYPEDSSYNNYPTATEGLNNEFLNFKRLQSAFQSENFLNWHVITDMFKNAFPFINWDFFPKT

(19) United States
(12) Patent Application Publication
(10) Pub. No.: US 2003/0211587 A1
(43) Pub. Date: Nov. 13, 2003

(54) Keptin-a novel keratinocyte-specific proteinase inhibitor

(75) Inventors: Kiyoshi Ariizumi, Plano, TX (US); Ponciano D. Cruz, Dallas, TX (US)

Correspondence Address: Steven L. Highlander, FULBRIGHT & JAWORSKI L.L.P., Suite 2400, 600 Congress Avenue, Austin, TX, 78701, US

(73) Assignee: Board of Regents, The University of Texas System

(21) Appl. No.: 10/141,530

(22) Filed: May 7, 2002

(51) Int. Cl. C07H 21/04; C12N 9/99; A61K 38/57; C12P 21/02; C12N 5/06; C07K 16/40

(52) U.S. Cl. 435/184; 435/69.2; 435/320.1; 435/325; 514/12; 536/23.2; 530/388.26; 435/338

(57) ABSTRACT

The present invention provides a novel keratinocyte-specific secreted protein identified as keptin that inhibits an inflammatory response and activity of proteinase, as well as methods for treating hyperproliferative inflammatory skin diseases using proteinase inhibitor keptin. The invention provides a therapeutic agent for treating psoriasis and squamous cell carcinomas and other inflammatory skin related diseases.

### BACKGROUND OF THE INVENTION

The government may own rights to the present invention pursuant to grant number P30AR41940-10.

1. Field of the Invention

The present invention relates generally to the fields of molecular biology, dermatology, cancer biology, and immunotherapy. More particularly, it concerns a nucleic acid segment encoding a novel keratinocyte-specific secreted protein, keptin for the treatment of hyperproliferative inflammatory skin disorders.

2. Description of Related Art

Keratinocytes play a critical role in hyperproliferative inflammatory skin diseases. These skin disorders include angiogenesis-related skin disorders, dermatomyositis—a inflammatory disease affecting the skin and muscles, carcinomas such as basal and squamous cell carcinoma and psoriasis.

Currently available treatments are generally directed towards reducing the rate of cell growth; keratolysis; anti-inflammatory action; inhibition of prostaglandin production; and inhibition of DNA synthesis. The most frequently used medications are corticosteroids, tar preparations, dithranol, vitamin D3 analogues (calcipotriol, calcitriol), ultraviolet light treatments and systemic drugs and therapies such as methotrexate, vitamin A derivatives (acitretin). These therapies have drawbacks in that they have high toxicity, low safety value and applicability, and are administered short term due to side effects. For example, inhibitory action on growth of cultured human epidermal keratinocyte cells by cyclosporin has been reported (Dykes et al., 1990). However, long term administration of cyclosporin causes renal lesion and discontinuation of this drug leads to recurrence of the disease state.

Overall, these medicaments have been shown to have insufficient therapeutic efficacy. Therefore, there is need for novel and alternative approaches for treating inflammatory skin disorders and hyperproliferative dermatological diseases as well as in microbial infections.

### SUMMARY OF THE INVENTION

The present invention overcomes the deficiencies in the prior art by providing a novel keratinocyte specific protein for use in the treatment of hyperproliferative nflammatory skin diseases. Therefore, in accordance with the present invention is provided herein an isolated nucleic acid segment encoding a keptin, or the complement of such a keptin. In particular embodiments the isolated nucleic acid encoding a keptin is human α keptin, human β keptin, mouse α keptin, mouse β keptin, mouse γ keptin, mouse δ keptin, or rat keptin.

In particular embodiments, the isolated nucleic acid segment encoding a keptin comprises the amino acid sequence of keptin selected from the group consisting of SEQ. ID. NO: 2, SEQ. ID. NO: 4, SEQ. ID. NO: 6, SEQ. ID. NO: 8, SEQ. ID. NO: 10, SEQ. ID. NO: 12 and SEQ ID NO: 14.

In another particular embodiment, the isolated nucleic acid segment encoding a keptin comprises the nucleotide sequence of keptin is selected from the group consisting of SEQ. ID. NO: 1, SEQ. ID. NO: 3, SEQ. ID. NO: 5, SEQ. ID. NO: 7, SEQ. ID. NO: 9, SEQ. ID. NO: 11 and SEQ ID NO: 13.

In yet another embodiment, the isolated nucleic acid encoding a keptin, further comprises a promoter such as a constitutive promoter, an inducible promoter, or a tissue specific promoter.

The isolated nucleic acid segment of the present invention, is comprised within an expression vector such as a viral or non-viral vector. The viral vector may further comprise an adenoviral vector, a herpesviral, a adeno-associated viral vector, a vaccinia viral vector, a polyoma viral vector or a retroviral vector. The non-viral vector may be comprised within a lipid membrane.

In other embodiments, the isolated nucleic acid segment of the present invention, further comprises one or more of a polyadenylation signal, an enhancer, an origin of replication, and a selectable marker gene.

The present invention embodies a method of expressing a keptin in a cell comprising introducing into the cell an expression vector comprising a nucleic acid segment encoding a keptin, and a promoter under conditions that support expression of the keptin.

In particular embodiments, the cell is a keratinocyte or a squamous cell carcinoma cell.

In yet another embodiment, the present invention provides a method of inhibiting an inflammatory response in a subject comprising providing to the subject an effective amount of a keptin, such that it inhibits protease activity in keratinocytes.

The present invention further embodies a method comprising administering the keptin polypeptide to the subject. In still a further embodiment, the present invention provides a method comprising administering an expression construct encoding the keptin polypeptide to the subject. The present invention further provides a method for administering a keptin percutaneously or subcutaneously.

In other embodiments the present invention provides a method of inhibiting an inflammatory response associated with a disease condition such as psoriasis or squamous cell carcinoma in a subject.

In yet another embodiment the present invention provides a method further comprising administering to a subject an anti-inflammatory drug selected from the group consisting of a COX inhibitor, a steroid, FK506 (tacrolimus), cyclosporin, and ASM (ascomycin).

In still yet another embodiment, the present invention provides a method of inhibiting microbial infection in a subject comprising providing to the subject an effective amount of a keptin, such that it inhibits protease activity in keratinocytes. In a further embodiment the present invention provides a method further comprising administering to a subject an anti-infectious agent selected from the group consisting of a proteinase inhibitor, an anti-fungal agent, an anti-bacterial agent, an anti-viral agent, and an antibiotic agent.

In particular embodiments, the present invention provides a monoclonal antibody that binds immunologically to a keptin; a hybridoma cell that produces a monoclonal antibody that binds immunologically to a keptin; and a polyclonal antibody preparation, antibodies of which bind immunologically to a keptin.

In still a further embodiment, the present invention provides an oligonucleotide of 10 to 50 bases in length, wherein the oligonucleotide comprises at least 10 consecutive bases from SEQ ID NO: 1, SEQ. ID. NO: 3, SEQ. ID. NO: 5, SEQ. ID. NO: 7, SEQ. ID. NO: 9, SEQ. ID. NO: 11 or SEQ ID NO: 13; and the oligonucleotide is 10, 15, 20, 25, 30, 35, 40, 45 or 50 bases in length.

In still a further embodiment, the present invention provides an oligonucleotide in which the number of consecutive bases from SEQ ID NO: 1, SEQ. ID. NO: 3, SEQ. ID. NO: 5, SEQ. ID. NO: 7, SEQ. ID. NO: 9, SEQ. ID. NO: 11 or SEQ ID NO: 13 is 10, 15, 20, 25, 30, 35, 40, 45 or 50 bases.

In still yet a further embodiment, the present invention provides a peptide of 10 to 50 residues in length, the peptide of which comprises at least 10 consecutive residues from SEQ ID NO: 2, SEQ. ID. NO: 4, SEQ. ID. NO: 6, SEQ. ID. NO: 8, SEQ. ID. NO: 10, SEQ. ID. NO: 12 or SEQ ID NO: 14; and is 10, 15, 20, 25, 30, 35, 40, 45 or 50 residues in length.

In still another embodiment, the number of consecutive residues from SEQ ID NO: 2, SEQ. ID. NO: 4, SEQ. ID. NO: 6, SEQ. ID. NO: 8, SEQ. ID. NO: 10, SEQ. ID. NO: 12 or SEQ ID NO: 14 is 10, 15, 20, 25, 30, 35, 40, 45 or 50.

The present invention embodies a method of inhibiting a proteinase comprising contacting to a subject an effective amount of a keptin, that inhibits protease activity in keratinocytes. The proteinase may be a serine proteinase or a cysteine proteinase and is located in a cell or in a cell-free environment.

The present invention also provides a pharmaceutical composition comprising a keptin or an expression construct encoding a keptin, and a pharmaceutically acceptable buffer, diluent or excipient.

### BRIEF DESCRIPTION OF THE DRAWINGS

The following drawings form part of the present specification and are included to further demonstrate certain aspects of the present invention. The invention may be better understood by reference to one or more of these drawings in combination with the detailed.

FIG. 1: Amino sequence of Keptin.

FIGS. 2A-2C: Anti-protease activity of His-Keptin. (FIG. 2A) Papain; (FIG. 2B) chymotrypsin, (FIG. 2C) ficin.

FIG. 3: Isoforms of Keptin.

### DESCRIPTION OF ILLUSTRATIVE EMBODIMENTS

I. The Present Invention

The present invention provides a soluble inhibitor designated as “keptin” as a medicament or therapy for hyperproliferative inflammatory skin diseases, having low toxicity, higher safety value, long term administration, and greater applicability. The present invention also provides keptin as an anti-infectious agent.

The invention, in particular, addresses psoriasis and squamous cell carcinoma as the proliferative skin disorders. However, the present invention encompasses a range of proliferative and/or inflammatory skin disorders or epidermal hyperplasias such as but not limited to: psoriasis, atopic dermatitis, nonspecific dermatitis, allergic contact dermatitis, primary irritant contact dermatitis, cutaneous basal cell carcinoma, cutaneous planocellular carcinoma, wart, lameliar ichthyosis, epidemolytic keratosis, solar induced precancerous keratosis, benign keratosis, ache, seborrheic dermatitis, keloids, pityriasis rubra pilaris (“PRP”), dermatomyositis, and angiogenesis-related skin disorders.

The present invention therefore, provides a novel and alternative method for treating hyperproliferative inflammatory skin disorders, and anti-infectious conditions, and overcomes the deficiencies in the art.

II. Keptin

The present invention provides a gene isolated from a cDNA library from human keratinocytes. The gene is expressed specifically by keratinocytes of epithelial tissues and encodes for a polypeptide of 99 amino acids identified herein as keptin, having a typical signal sequence of 22 amino acids. The secreted form of keptin is 77 amino acids and is a novel protein in that it has no homology to known molecules. Keptin is a small molecular weight protein of 12.5 kDa containing no cysteine residues which suggests the formation of a three dimensional structure by leucine-based hydrophobic interactions.

Keptin is further characterized by: 1) three FLN repeats each consisting of 15 or 16 amino acids with 33% homology; 2) six leucine residues aligned periodically, a motif responsible for its dimerization; 3) it is expressed exclusively by differentiated keratinocytes in the granular layer of normal human epidermis, indicating its localization in the cornified cell envelope, an essential component in the epidermal barrier; and 4) it can inhibit the activities of cysteine proteinases such as papain and serine proteinases such as chymotrypsin. Keptin is thus further defined as a keratinocyte-specific secreted inhibitor of proteinases which are produced by tissue damage (e.g., infection, inflammation, wound, chemicals, and UV radiation) and either modulate the function of keratinocytes or are lethal to them. Keptin, therefore is further defined as a novel keratinocyte-specific secreted protein involved in cricumventing the damaging effects of proteinases.

As used herein, an “amino acid residue” refers to any naturally occurring amino acid, any amino acid derivative or any amino acid mimic known in the art, including modified or unusual amino acids. In certain embodiments, the residues of the protein or peptide are sequential, without any non-amino acid interrupting the sequence of amino acid residues. In other embodiments, the sequence may comprise one or more non-amino acid moieties. In particular embodiments, the sequence of residues of the protein or peptide may be interrupted by one or more non-amino acid moieties.

Proteins or peptides may be made by any technique known to those of skill in the art, including the expression of proteins, polypeptides or peptides through standard molecular biological techniques, the isolation of proteins or peptides from natural sources, or the chemical synthesis of proteins or peptides. Because of their relatively small size, targeting peptides of the current invention can be synthesized by conventional techniques (see, for example, Stewart and Young, (1984); Tam et al. (1983); Merrifield, (1986); and Barany and Merrifield (1979), each incorporated herein by reference. Alternatively, various commercial preparations of proteins, polypeptides and peptides are known to those of skill in the art. The sequence of a keptin polypeptide will substantially correspond to a contiguous portion of that shown in SEQ ID NO: 2, SEQ. ID. NO: 4, SEQ. ID. NO: 6, SEQ. ID. NO: 8, SEQ. ID. NO: 10, SEQ. ID. NO: 12 or SEQ ID NO: 14, and have relatively few amino acids that are not identical to, or a biologically functional equivalent of, the amino acids shown in SEQ ID NO: 2, SEQ. ID. NO: 4, SEQ. ID. NO: 6, SEQ. ID. NO: 8, SEQ. ID. NO: 10, SEQ. ID. NO: 12 or SEQ ID NO: 14. The term “biologically functional equivalent” is well understood in the art and is further defined in detail herein.

Accordingly, sequences that have between about 70% and about 80%; or more preferably, between about 81% and about 90%; or even more preferably, between about 91% and about 99%; of amino acids that are identical or functionally equivalent to the amino acids of SEQ ID NO: 2, SEQ. ID. NO: 4, SEQ. ID. NO: 6, SEQ. ID. NO: 8, SEQ. ID. NO: 10, SEQ. ID. NO: 12 or SEQ ID NO: 14, will be sequences that are “essentially as set forth in SEQ ID NO: 2, SEQ. ID. NO: 4, SEQ. ID. NO: 6, SEQ. ID. NO: 8, SEQ. ID. NO: 10, SEQ. ID. NO: 12 or SEQ ID NO: 14.”

It is contemplated by the inventors that the proteins of the present invention may further employ amino acid sequence variants such as substitutional, insertional or deletion variants. Deletion variants lack one or more residues of the native protein. Insertional mutants typically involve the addition of material at a non-terminal point in the polypeptide. Substitutions are changes to an existing amino acid. These sequence variants may generate truncations, point mutations, and frameshift mutations. As is known to one skilled in the art, synthetic peptides can be generated by these mutations.

It also will be understood that amino acid may include additional residues, such as additional N- or C-terminal amino acids, and yet still be essentially as set forth in one of the sequences disclosed herein, so long as the sequence meets the criteria set forth above, including the maintenance of biological activity.

The following is a discussion based upon changing the amino acids of a protein, such as a keptin protein, to create a mutated, truncated, or modified protein. For example, certain amino acids may be substituted for other amino acids in the keptin protein, resulting in a greater binding specificity, cell uptake and enhancement of an immune response. Since it is the interactive capacity and nature of a protein that defines that protein's biological functional activity, certain amino acid substitutions can be made in a protein sequence, and in its underlying DNA coding sequence, thereby producing a mutated, truncated or modified protein. It is thus contemplated by the inventors that various changes may be made in the DNA sequences of genes as is discussed below.

In making such changes, the hydropathic index of amino acids may be considered. The importance of the hydropathic amino acid index in conferring interactive biologic function on a protein is generally understood in the art (Kyte & Doolittle, 1982). It is accepted that the relative hydropathic character of the amino acid contributes to the secondary structure of the resultant protein, which in turn defines the interaction of the protein with other molecules, for example, enzymes, substrates, receptors, DNA, antibodies, antigens, and the like.

It also is understood in the art that for amino acids positioned in the homologous region of nucleotide and encodes for the protein or polypeptide, the substitution of pairs of homologous and non-homologous amino acids can be made effectively on the basis of polarity. Non-homologous amino acids may be conservatively substituted with a member of the same polarity group as defined below: basic amino acids: arginine (+3.0), lysine (+3.0), and histidine (−0.5); acidic amino acids: aspartate (+3.0±1), glutamate (+3.0±1), asparagine (+0.2), and glutamine (+0.2); hydrophilic, nonionic amino acids: serine (+0.3), asparagine (+0.2), glutamine (+0.2), and threonine (−0.4), sulfur containing amino acids: cysteine (−1.0) and methionine (−1.3); hydrophobic, nonaromatic amino acids: valine (−1.5), leucine (−1.8), isoleucine (−1.8), proline (−0.5±1), alanine (−0.5), and glycine (0); hydrophobic, aromatic amino acids: tryptophan (−3.4), phenylalanine (−2.5), and tyrosine (−2.3).

It is understood that an amino acid can be substituted for another having a similar hydrophilicity and produce a biologically or immunologically modified protein. In such changes, the substitution of amino acids whose hydrophilicity values are within ±2 is preferred, those that are within ±1 are particularly preferred, and those within ±0.5 are even more particularly preferred.

As outlined above, amino acid substitutions generally are based on the relative similarity of the amino acid side-chain substituents, for example, their hydrophobicity, hydrophilicity, charge, size, and the like. Exemplary substitutions that take into consideration the various foregoing characteristics are well known to those of skill in the art and include: arginine and lysine; glutamate and aspartate; serine and threonine; glutamine and asparagine; and valine, leucine and isoleucine.

The present invention also embodies the use of peptide mimetics for the preparation of polypeptides (see e.g., Johnson, 1993) having many of the natural properties of the keptin protein, but with altered and/or improved characteristics.

III. Anti-Keptin Antibodies

The present invention embodiments, at least one antibody, for example, an antibody against a keptin. As used herein, the term “antibody” is intended to refer broadly to any immunologic binding agent such as IgG, IgM, IgA, IgD and IgE. Generally, IgG and/or IgM are preferred because they are the most common antibodies in the physiological situation and because they are most easily made in a laboratory setting.

The term “antibody” is used to refer to any antibody-like molecule that has an antigen binding region, and includes antibody fragments such as Fab′, Fab, F(ab′)₂, single domain antibodies (DABs), Fv, scFv (single chain Fv), and the like. Monoclonal and polyclonal antibodies specific for a keptin protein and related proteins that are expressed in keratinocytes, will have utilities in several applications. Additionally, these antibodies may be linked to therapuetic agents as described herein, and administered to a subject. The techniques for preparing and using various antibodies and antibody-based constructs and fragments are well known in the art (See, e.g., Harlow et al., 1988; and U.S. Pat. No. 4,196,265 each incorporated herein by reference).

IV. Nucleic Acid Segments Encoding Keptin

The present invention concerns nucleic acid segments, isolatable from cells, that are free from total genomic DNA and that are capable of expressing all or part of a protein or polypeptide such as a keptin. The nucleic acid may encode a peptide or polypeptide containing all or part of the keptin amino acid sequence.

As used herein, the term “nucleic acid segment” refers to a DNA molecule that has been isolated free of total genomic DNA of a particular species. Therefore, a nucleic acid segment encoding a keptin refers to a nucleic acid segment that contains wild-type, polymorphic, or mutant polypeptide-coding sequences yet is isolated away from, or purified free from, total mammalian or human genomic DNA. Included within the term “nucleic acid segment” are a polypeptide(s), nucleic acid segments smaller than a polypeptide, and recombinant vectors, including, for example, plasmids, cosmids, phage, viruses, and the like.

The term “cDNA” is intended to refer to DNA prepared using messenger RNA (mRNA) as template. The advantage of using a cDNA, as opposed to genomic DNA or DNA polymerized from a genomic, non- or partially-processed RNA template, is that the cDNA primarily contains coding sequences of the corresponding protein. There may be times when the full or partial genomic sequence is preferred, such as where the non-coding regions are required for optimal expression or where non-coding regions such as introns are to be targeted in an antisense strategy.

A nucleic acid segment encoding all or part of a native or modified polypeptide may contain a contiguous nucleic acid sequence encoding all or a portion of such a polypeptide of the following lengths: about 10, about 20, about 30, about 40, about 50, about 60, about 70, about 80, about 90, or about 100 or more nucleotides, nucleosides, or base pairs.

The nucleic acid segments used in the present invention, regardless of the length of the coding sequence itself, may be combined with other nucleic acid sequences, such as promoters, enhancers polyadenylation signals, origin of replication, and a selectable marker gene, as well as other coding segments, and the like (all as are known to those of ordinary skill in the art), such that their overall length may vary considerably.

The term oligonucleotide refers to at least one molecule of between about 3 and about 100 nucleobases in length. These definitions generally refer to at least one single-stranded molecule, but in specific embodiments will also encompass at least one additional strand that is partially, substantially or fully complementary to the at least one single-stranded molecule. Thus, a nucleic acid may encompass at least one double-stranded molecule or at least one triple-stranded molecule that comprises one or more complementary strand(s) or “complement(s)” of a particular sequence comprising a strand of the molecule.

It is contemplated that the nucleic acid constructs of the present invention may encode full-length polypeptide from any source or encode a truncated version of the polypeptide, for example a truncated keptin polypeptide, such that the transcript of the coding region represents the truncated version. The truncated transcript may then be translated into a truncated protein. Alternatively, a nucleic acid sequence may encode a full-length polypeptide sequence with additional heterologous coding sequences, for example to allow for purification of the polypeptide, transport, secretion, post-translational modification, or for therapeutic benefits such as targeting or efficacy.

It is contemplated that the nucleic acid constructs of the present invention may regulate gene expression of an immunogenic polypeptide. A nucleic acid segment may regulate the expression of a full-length polypeptide sequence with additional heterologous coding sequences, for example to allow for therapeutic benefits such as targeting or efficacy.

In a non-limiting example, one or more nucleic acid constructs may be prepared that include a contiguous stretch of nucleotides identical to or complementary to a particular gene, such as the human keptin gene (SEQ ID NO: 3). Such a nucleic acid construct may be at least 20, 30, 40, 50, 60, 70, 80, 90, 100, 110, 120, 130, 140, 150, 160, 170, 180, 190, 200, 250, 300, 400, 500, 600, 700, 800, 900, 1,000, 2,000, 3,000, 4,000, 5,000, 6,000, 7,000, 8,000, 9,000, 10,000, 15,000, 20,000, 30,000, 50,000, 100,000, 250,000, 500,000, 750,000, to at least 1,000,000 nucleotides in length, as well as constructs of greater size, up to and including chromosomal sizes (including all intermediate lengths and intermediate ranges), given the advent of nucleic acids constructs such as a yeast artificial chromosome are known to those of ordinary skill in the art. It will be readily understood that “intermediate lengths” and “intermediate ranges,” as used herein, means any length or range including or between the quoted values (i.e., all integers including and between such values).

In certain other embodiments, the invention concerns isolated nucleic acid segments and recombinant vectors that include within their sequence a contiguous nucleic acid sequence from that shown in SEQ ID NO: 1, SEQ. ID. NO: 3, SEQ. ID. NO: 5, SEQ. ID. NO: 7, SEQ. ID. NO: 9, SEQ. ID. NO: 11 or SEQ ID NO: 13. This definition is used in the same sense as described above and means that the nucleic acid sequence substantially corresponds to a contiguous portion of that shown in SEQ ID NO: 1, 3, 5, 7, 9, 11 or 13 and has relatively few codons that are not identical, or functionally equivalent, to the codons of SEQ ID NO: 1, 3, 5, 7, 9, 11 or 13. The term “functionally equivalent codon” is used herein to refer to codons that encode the same amino acid, such as the six codons for arginine or serine, and also refers to codons that encode biologically equivalent amino acids, as is known to those of skill in the art.

The various probes and primers designed around the nucleotide sequences of the present invention may be of any length. By assigning numeric values to a sequence, for example, the first residue is 1, the second residue is 2, etc., an algorithm defining all primers can be proposed:

n to n+y

where n is an integer from 1 to the last number of the sequence and y is the length of the primer minus one, where n+y does not exceed the last number of the sequence. Thus, for a 10-mer, the probes correspond to bases 1 to 10, 2 to 11, 3 to 12 . . . and so on. For a 15-mer, the probes correspond to bases 1 to 15, 2 to 16, 3 to 17 . . . and so on. For a 20-mer, the probes correspond to bases 1 to 20, 2 to 21, 3 to 22 . . . and so on.

It also will be understood that this invention is not limited to the particular nucleic acid sequences of SEQ ID NO: 1, 3, 5, 7, 9, 11 or 13 and amino acid sequences of SEQ ID NO: 2, SEQ. ID. NO: 4, SEQ. ID. NO: 6, SEQ. ID. NO: 8, SEQ. ID. NO: 10, SEQ. ID. NO: 12 or SEQ ID NO: 14. Recombinant vectors and isolated DNA segments may therefore variously include the keptin-coding regions themselves, coding regions bearing selected alterations or modifications in the basic coding region, or they may encode larger polypeptides that nevertheless include keptin-coding regions or may encode biologically functional equivalent proteins or peptides that have variant amino acids sequences.

The nucleic acid segments of the present invention encompass biologically functional equivalent keptin proteins and peptides. Such sequences may arise as a consequence of codon redundancy and functional equivalency that are known to occur naturally within nucleic acid sequences and the proteins thus encoded. Alternatively, functionally equivalent proteins or peptides may be created via the application of recombinant DNA technology, in which changes in the protein structure may be engineered, based on considerations of the properties of the amino acids being exchanged. Changes designed by man may be introduced through the application of site-directed mutagenesis techniques, e.g., to introduce improvements to the antigenicity of the protein.

If desired, one also may prepare fusion proteins and peptides, e.g., where the keptin-coding regions are aligned within the same expression unit with other proteins or peptides having desired functions, such as for purification or immunodetection purposes (e.g., proteins that may be purified by affinity chromatography and enzyme label coding regions, respectively).

Encompassed by certain embodiments of the present invention are nucleic acid segments encoding relatively small peptides, such as, for example, peptides of from about 10 to about 100 amino acids in length, and more preferably, of from about 10 to about 80 amino acids in length; and also larger polypeptides up to and including proteins corresponding to the full-length sequences set forth in SEQ ID NO: 2, 4, 6, 8, 10, 12 or 14, or to specific fragments of SEQ ID NO: 1, 3, 5, 7, 9, 11 or 13.

A. Promoters

The present invention may also involve expression of keptin from a keptin-encoding nucleic acid. This requires the presence of a promoter operably linked to the keptin-coding region. A promoter generally comprises a nucleic acid sequence that functions to position the start site for RNA synthesis. A promoter may or may not be used in conjunction with an enhancer, which refers to a cis-acting regulatory sequence involved in the transcriptional activation of a nucleic acid sequence. In the present invention, a nucleic acid encoding a keptin comprises a promoter such as a tissue specific promoter, or a constitutive promoter, or an inducible promoter.

A promoter in the context of the present invention may be one naturally associated with a gene or sequence, as may be obtained by isolating the 5′ non-coding sequences located upstream of the coding segment and/or exon. Such a promoter can be referred to as “endogenous.” Similarly, an enhancer may be one naturally associated with a nucleic acid sequence, located either downstream or upstream of that sequence. Alternatively, certain advantages will be gained by positioning the coding nucleic acid segment under the control of a recombinant or heterologous promoter or enhancer, which refers to a promoter or enhancer, that is not normally associated with a nucleic acid sequence in its natural environment. Such promoters or enhancers may include promoters or enhancers of other genes, and promoters or enhancers isolated from any other prokaryotic, viral, or eukaryotic cell, and promoters or enhancers not “naturally occurring,” i.e., containing different elements of different transcriptional regulatory regions, and/or mutations that alter expression. In addition to producing nucleic acid sequences of promoters and enhancers synthetically, sequences may be produced using recombinant cloning and/or nucleic acid amplification technology, including PCR™, in connection with the compositions disclosed herein (see U.S. Pat. No. 4,683,202, U.S. Pat. No. 5,928,906, each incorporated herein by reference). Furthermore, it is contemplated the control sequences that direct transcription and/or expression of sequences within non-nuclear organelles such as mitochondria, chloroplasts, and the like, can be employed as well.

Those of skill in the art of molecular biology generally know the use of promoters, enhancers, and cell type combinations for protein expression, for example, see Sambrook et al. (2000), incorporated herein by reference.

The present invention also contemplates the use of tissue specific promoters which include but are not limited to: keratin 1, 4, 6, 17 and 19, EBV LD-2, Fas ligand, Myc-regulated promoter MAGE-1, VEGF, COX-2, IL-10, and tryosinase. Other promoters that may be employed with the present invention are constitutive and inducible promoters as are well known to those of skill in the art. These include but are not limited to: IL-1 alpha and beta, IL-8, IL-13, interferon, SV40, CMV, and adenovirus. Additionally any promoter/enhancer combination (as per the Eukaryotic Promoter Data Base EPDB) could also be used to drive expression of structural genes encoding oligosaccharide processing enzymes, protein folding accessory proteins, selectable marker proteins or a heterologous protein of interest.

B. Origins of Replication/Polyadenylation Signal

In expression, particularly eukaryotic expression, one will typically include a polyadenylation signal to effect proper polyadenylation of the transcript. The nature of the polyadenylation signal is not believed to be crucial to the successful practice of the invention. Polyadenylation signals include the SV40 polyadenylation signal and the bovine growth hormone polyadenylation signal, known to function well in various target cells. Polyadenylation may increase the stability of the transcript or may facilitate cytoplasmic transport.

In order to propagate a vector in a host cell, it may contain one or more origins of replication sites (often termed “ori”), which is a specific nucleic acid sequence at which replication is initiated. Alternatively an autonomously replicating sequence (ARS) can be employed if the host cell is yeast.

C. Selectable and Screenable Markers

In certain embodiments of the invention, cells containing a nucleic acid construct of the present invention may be identified in vitro or in vivo by including a marker in the expression vector. Such markers would confer an identifiable change to the cell permitting easy identification of cells containing the expression vector, and are well known to one of skill in the art. Usually the inclusion of a drug selection marker aids in the cloning and identification of transformants, for example, genes that confer resistance to neomycin, puromycin, hygromycin, DHFR, GPT, zeocin and histidinol are useful selectable markers. In addition to markers conferring a phenotype that allows for the discrimination of transformants based on the implementation of conditions, other types of markers including screenable markers such as GFP, whose basis is colorimetric analysis, are also contemplated. Alternatively, screenable enzymes such as herpes simplex virus thymidine kinase (tk) or chloramphenicol acetyltransferase (CAT) may be utilized. Immunologic markers may also be employed. The selectable marker used is not believed to be important, so long as it is capable of being expressed simultaneously with the nucleic acid encoding a gene product.

V. Delivery of a Nucleic Acids

Two broad approaches have been used to employ vectors deliver nucleic acids to cells, namely viral vectors and non-viral vectors. As by methods described herein and as is known to the skilled artisan, expression vectors may be constructed to deliver nucleic acids segments encoding a keptin of the present invention to a organelle, cell, tissue, or a subject. Such vectors comprising a keptin may be used in a variety of manner consistent with the invention, including in screening assay and genetic immunization protocols.

A vector in the context of the present invention refers to a carrier nucleic acid molecule into which a nucleic acid sequence of the present invention may be inserted for introduction into a cell and thereby replicated. A nucleic acid sequence can be exogenous, which means that it is foreign to the cell into which the vector is being introduced; or that the sequence is homologous to a sequence in the cell but positioned within the host cell nucleic acid in which the sequence is ordinarily not found. Vectors include plasmids; cosmids; viruses such as bacteriophage, animal viruses, and plant viruses; and artificial chromosomes (e.g., YACs); and synthetic vectors. One of ordinary skill in the art would be well equipped to construct any number of vectors through standard recombinant techniques as described in Maniatis et al., 1990 and Ausubel et al., 1994, incorporated herein by reference.

Viral vectors may be derived from viruses know to those of skill in the art, for example, bacteriophage, animal and plant virus, including but not limited to, adenovirus, vaccinia virus (Ridgeway, 1988; Baichwal and Sugden, 1986; Coupar et al., 1988) adeno-associated virus (AAV) (Ridgeway, 1988; Baichwal and Sugden, 1986; Hermonat and Muzycska, 1984) retrovirus and herpesvirus and offer several features for use in gene transfer into various mammalian cells (Friedmann, 1989; Ridgeway, 1988; Baichwal and Sugden, 1986; Coupar et al, 1988; Horwich et al., 1990). One of skill in the art would be well equipped to construct a vector through standard recombinant techniques as described in Sambrook et al., 2001, Maniatis et al., 1990 and Ausubel et al., 1994, incorporated herein by reference. The present invention may also employ non-viral vectors.

An expression vector refers to any type of genetic construct comprising a nucleic acid coding for a RNA capable of being transcribed. In the context of the present specification, expression vectors will typically comprise a nucleic acid segment encoding a keptin as described herein. In some cases, RNA molecules are then translated into a protein, polypeptide, or peptide. In other cases, these sequences are not translated, as in the case of antisense molecules or ribozymes production. Expression vectors can contain a variety of control sequences, which refer to nucleic acid sequences necessary for the transcription and possibly translation of an operably linked coding sequence in a particular host cell. In addition to control sequences that govern transcription and translation, vectors and expression vectors may contain nucleic acid sequences that serve other functions as well, and are described herein.

Non-viral vectors, such as plasmids and cosmids, require suitable method for delivery into cells. Such methods include, but are not limited to direct delivery of DNA by: injection (U.S. Pat. Nos. 5,994,624, 5,981,274, 5,945,100, 5,780,448, 5,736,524, 5,702,932, 5,656,610, 5,589,466 and 5,580,859, each incorporated herein by reference), microinjection (Harlan and Weintraub, 1985; U.S. Pat. No. 5,789,215, incorporated herein by reference); electroporation (U.S. Pat. No. 5,384,253, incorporated herein by reference; Tur-Kaspa et al, 1986; Potter et al., 1984); calcium phosphate precipitation (Graham and Van Der Eb, 1973; Chen and Okayama, 1987; Rippe et al, 1990); using DEAE-dextran followed by polyethylene glycol (Gopal, 1985); direct sonic loading (Fechheimer et aL., 1987); by liposome mediated transfection (Nicolau and Sene, 1982; Fraley et al., 1979; Nicolau et al., 1987; Wong et al., 1980; Kaneda et al., 1989; Kato et al, 1991); receptor-mediated transfection (Wu and Wu, 1987; Wu and Wu, 1988); microprojectile bombardment (PCT Application Nos. WO 94/09699 and 95/06128; U.S. Pat. Nos. 5,610,042; 5,322,783, 5,563,055, 5,550,318, 5,538,877 and 5,538,880, and each incorporated herein by reference); agitation with silicon carbide fibers (Kaeppler et al., 1990; U.S. Pat. Nos. 5,302,523 and 5,464,765, each incorporated herein by reference); desiccation/inhibition-mediated DNA uptake (Potrykus et al., 1985), and any combination of such methods. Through the application of techniques such as these, organelle(s), cell(s), tissue(s) or organism(s) may be stably or transiently transformed. In certain embodiments, acceleration methods are preferred and include, for example, microprojectile bombardment or inhalation methods.

Also in context of the present invention, topical delivery of a nucleic acid segment encoding a keptin to the skin may further comprise vesicles such as liposomes, niosomes and transferosomes thereby enhancing topical and transdermal delivery. Cationic lipids may also be used to deliver negatively charged nucleic acids. Sonophoresis or phonophoresis which involves the use of ultrasound to delivery the nucleic acid of interest, may also be employed for transdermal delivery. lonotophoresis which consists of applying a low electric filed for a period of time to the skin may also be applied in delivering the nucleic acid of interest to the skin.

VII. Pharmaceutical Formulations, Delivery, and Treatment Regimens

In particular embodiments of the present invention, a method of treatment for a hyperproliferative inflammatory skin disease, such as psoriasis, or skin cancer disease by the delivery of a keptin is contemplated. Hyperproliferative inflammatory skin diseases that are most likely to be treated in the present invention are those that result from tissue damage, infection, cancer, or proteinases. Examples of hyperproliferative inflammatory skin diseases contemplated for treatment include a range of proliferative and/or inflammatory skin disorders or epidermal hyperplasias such as, but not limited to: psoriasis, atopic dermatitis, nonspecific dermatitis, allergic contact dermatitis, primary irritant contact dermatitis, cutaneous basal cell carcinoma, cutaneous planocellular carcinoma, wart, lameliar ichthyosis, epidemolytic keratosis, solar induced precancerous keratosis, benign keratosis, ache, seborrheic dermatitis, keloids, pityriasis rubra pilaris (“PRP”), dermatomyositis, angiogenesis-related skin disorders, erysipleas, and erythroderma.

A. Administration

To inhibit an inflammatory response in a hyperproliferative inflammatory skin disease or to inhibit proteinase activity in keratinocytes using the methods and compositions of the present invention, one would generally contact a hyperproliferative inflammatory cell with the therapeutic compound such as a keptin polypeptide or an expression construct encoding a keptin polypeptide. The preferred method for the delivery of an expression construct encoding all or part of a keptin protein to hyperproliferative inflammatory skin disease cells in the present invention, is subcutaneously or precutaneously. However, the pharmaceutical compositions disclosed herein may alternatively be administered parenterally, intradermally, intramuscularly, transdernally, perfusion, lavage, direct injection, intratumoral administration, and oral administration and formulation as described in U.S. Pat. Nos. 5,543,158; 5,641,515; 5,399,363 (each specifically incorporated herein by reference in its entirety). Injection of nucleic acid constructs of the present invention may be delivered by syringe or any other method used for injection of a solution, as long as the expression construct can pass through the particular gauge of needle required for injection. A novel needleless injection system (U.S. Pat. No. 5,846,233); or a syringe system for use in gene therapy (U.S. Pat. No. 5,846,225), all as incorporated herein by reference, may be employed in the present invention.

B. Compositions and Formulations

In the context of the present invention, conventional formulations such as capsules, granules for oral administration, suppositories, percutaneous absorption preparation can be prepared. The nucleic acid segment encoding a keptin may be in aqueous solution or in conjunction with a cream, ointment, oil or other suitable carrier and/or diluent. Solutions of the active compounds as free base or pharmacologically acceptable salts may be prepared in water suitably mixed with a surfactant, such as hydroxypropylcellulose. Dispersions may also be prepared in glycerol, liquid polyethylene glycols, and mixtures thereof and in oils. Under ordinary conditions of storage and use, these preparations contain a preservative to prevent the growth of microorganisms. The pharmaceutical forms suitable for injectable use include sterile aqueous solutions or dispersions and sterile powders for the extemporaneous preparation of sterile injectable solutions or dispersions (U.S. Pat. No. 5,466,468, specifically incorporated herein by reference in its entirety). In all cases the form must be sterile and must be fluid to the extent that easy syringability exists. Composition(s) of absorption delay agents(aluminum monostearate and gelatin) may also be used. It must be stable under the conditions of manufacture and storage and must be preserved against the contaminating action of microorganisms, such as bacteria and fungi. The carrier can be a solvent or dispersion medium containing, for example, water, ethanol, polyol (e.g., glycerol, propylene glycol, and liquid polyethylene glycol, and the like), suitable mixtures thereof, and/or vegetable oils. Proper fluidity may be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like.

For parenteral administration in an aqueous solution, for example, the solution should be suitably buffered if necessary and the liquid diluent first rendered isotonic with sufficient saline or glucose (see for example, “Remington's Pharmaceutical Sciences” 15th Edition, pages 1035-1038 and 1570-1580). These particular aqueous solutions are especially suitable for subcutaneous, intramuscular, and intratumoral administration. In this connection, sterile aqueous media that may be employed will be known to those of skill in the art in light of the present disclosure. Variation in dosage will necessarily occur depending on the condition of the subject being treated; the severity of the condition, and will be determined by the person administering the dose. For human administration, preparations should meet sterility, pyrogenicity, general safety and purity standards as required by FDA Office of Biologics standards.

The compositions of the present invention may be formulated in a neutral or salt form. Pharmaceutically-acceptable salts, include the acid addition salts (formed with the free amino groups of the protein) and which are formed with inorganic acids; or salts (formed with the free carboxyl groups) derived from inorganic bases as is known to those of ordinary skill in the art.

As used herein, “carrier” includes any and all solvents, dispersion media, vehicles, coatings, diluents, antibacterial and antifungal agents, isotonic and absorption delaying agents, buffers, carrier solutions, suspensions, colloids, and the like. The use of such media and agents for pharmaceutical active substances is well known in the art. Except insofar as any conventional media or agent is incompatible with the active ingredient, its use in the therapeutic compositions is contemplated. Supplementary active ingredients can also be incorporated into the compositions.

The phrase “pharmaceutically-acceptable” or “pharmacologically-acceptable” refers to molecular entities and compositions that do not produce an allergic or similar untoward reaction when administered to a human. The preparation of an aqueous composition that contains a protein as an active ingredient is well understood in the art.

C. Combination Treatments

The compounds and methods of the present invention may be used in the context of hyperproliferative inflammatory diseases/conditions which include but are not limited to psoriasis or skin cancers. In order to increase the effectiveness of a treatment with the compositions of the present invention, a nucleic acid encoding a keptin, or a recombinant keptin, or an expression construct coding therefor, it may be desirable to combine these compositions with other agents effective in the treatment of those diseases and conditions. For example, the treatment of an inflammatory skin disease may be implemented with therapeutic compounds of the present invention and conventional agents or therapies, or may employ treatment of the particular disease or condition.

Administration of a nucleic acid encoding a keptin of the present invention to a subject will follow general protocols for the administration of that particular secondary therapy, taking into account the toxicity, if any, of the therapeutic treatment. It is expected that the treatment cycles would be repeated as necessary. It also is contemplated that various standard therapies, as well as surgical intervention, may be applied in combination with the therapy of the disease or condition.

Various combinations of times of treatment may be used. For example, the therapies involving a nucleic acid encoding a keptin may precede or follow treatment with other agents by intervals ranging from minutes to weeks. In embodiments where the other agent and a nucleic acid encoding a keptin are applied separately to the cell, one would generally ensure that a significant period of time did not expire between the time of each delivery, such that the agent and the a nucleic acid encoding a keptin would still be able to exert an advantageously combined effect on the cell. In such instances, it is contemplated that one may contact the cell with both modalities within about 12-24 h of each other and, more preferably, within about 6-12 h of each other. In some situations, it may be desirable to extend the time period for treatment, depending on the severity of the condition, where administration may range from hourly, daily, weekly or monthly, or other suitable time intervals.

Various combinations may be employed; for example, a keptin is “A” and the secondary hyperproliferative inflammatory skin disease therapy is “B”:

| | | | | | | | |
|---|---|---|---|---|---|---|---|
| A/B/A | B/A/B | B/B/A | A/A/B | A/B/B | B/A/A | A/B/B/B | B/A/B/B |

| | | | | | |
|---|---|---|---|---|---|
| B/B/B/A | B/B/A/B | A/A/B/B | A/B/A/B | A/B/B/A | B/B/A/A |
| B/A/B/A | B/A/A/B | A/A/A/B | B/A/A/A | A/B/A/A | A/A/B/A |

Administration of the therapeutic expression constructs of the present invention to a patient will follow general protocols for the administration of that particular secondary therapy, taking into account the toxicity, if any, of keptin treatment. It is expected that the treatment cycles would be repeated as necessary. It also is contemplated that various standard therapies, as well as surgical intervention, may be applied in combination with the described hyperproliferative inflammatory skin disease therapies. For example, therapeutic efforts in psoriasis are aimed at decreasing the proliferative rate of the epidermis either by direct action on cell division, or through agents that reduce the inflammatory response or vascular permeability. These agents, such as calcipotriol (D-vitamin derivative), dithranol or corticosteroids may be used with the present invention to further decrease the disease state.

1. Proteinase and Other Inhibitors

The nucleic acid encoding a keptin of the present invention may be used in combination with other inhibitors such as the proteinease inhibitors SLP1 (secretory protease inhibitors which inhibits leukocyte derived proteinases and act as an initial defense mechanism in cuteaneous injury, disrupting the induction of synthesis of proinflammatory mediators in monocytes and macrophages. Additionally, the present invention may be used with SKALP (skin derived antileucoproteinase) also known as elafin, and involved in regulating cutaneous inflammatory processes.

It is also contemplated growth factor inhibitors may be employed with the present invention in treating hyperproliferative inflammatory skin diseases. Such therapies may include but are not limited to using epidermal cell growth inhibitors, insulin growth factor inhibitors (IGF-1), keratinocyte-derived nerve growth factor inhibitors, and transforming growth factor beta (TGF-β).

2. Anticancer Agents

The nucleic acid segment encoding a keptin of the present invention may also be used in treating carcinomas of the skin such as basal cell carcinoma, squamous cell carcinoma, melanoma, and angiogenesis related inflammatory skin diseases. Therefore, in specific embodiments the present invention may be used in combination with other anti-cancer therapies which include biological agents (biotherapy), chemotherapy agents, and radiotherapy agents, as is known to those of skill in the art. An anti-cancer agent is capable of negatively affecting cancer in a subject. In the context of the present invention, it is contemplated that a therapuetic agent containing a nucleic acid segment encoding a keptin, can be used in conjunction with chemotherapeutic, radiotherapeutic, immunotherapeutic or other biological intervention, in addition to other pro-apoptotic or cell cycle regulating agents or surgery. Thus, it is contemplated that one or more anti-cancer therapies, as is known to the one of skill in the art, may be employed with the nucleic acid segment encoding a keptin as described herein. Some examples of anticancer gents include but are not limited to: 5-fluorouracil; alpha and gamma interferon; mitotic inhibitors which include, for example, docetaxel, etoposide (VP16), teniposide, paclitaxel, methotrexate, taxol, vinblastine, vincristine, and vinorelbine; and any of the other combinations of therapies described herein.

3. Retinoids and Other Steroids

The present invention may also be used in combination with retinoids, particularly eretrinate, either alone or in combination with ultraviolet light, as an effective treatment for hyperproliferative inflammatory diseases such as psoriasis. Retinoic acid isomers (all-trans- and 11-cis isomers); topical retinoids: Tretinoin (Retin-A) Cream or Gel, Benzoyl peroxide gel, Adapalene (Differin) gel, Clindamycin (Cleocin T) gel, Azelaic acid (Azelex); tetracycline HCl; AGN190168 (tazarotene); and vitamin A derivatives (acitretin), in combination with the present invention, may be used as a treatment for hyperproliferative inflammatory skin disorders.

Adrenocorticosteroids/corticosteroids such as prednisone, dexamethasone, hydroxychloroquine (Plaquenil), oral corticosteroids (prednisone), azathioprine (Imuran), mycophenolate mofetil (Cell Cept), methotrexate, or cyclophosphamide (Cytoxan) may also be used in combination with the present invention either alone or with other steriod family members such as the retinoids.

4. Immunosuppressive Agents

Immunosuppressants such as tacrolimus (FK506), rapamycin, cyclosporin A (Neoral), ascomycin, and ascomycin derivatives such as SDZ ASM 981 may also be used with the nucleic acid segment of the present invention in the treatment of dermatological or hyperproliferative inflammatory diseases/disorders. Other immunosuppressive agents such as mizoribine (Bredinin™), prednisone, azathioprine (Imuran), mycophenolate mofetil (CellCept) or cyclophosphamide (Cytoxan) may also be used with the nucleic segment encoding a keptin in the present invention. Other immunosuppressive agents may also be employed with the present invention.

5. Phototherapy (UV Irradiation) and Photodynamic Therapy

Hyperproliferative inflammatory skin disease may also be treated with photochemotherapy. This method consists of administering psoralen in the form of an external or internal preparation and applying longwave ultraviolet rays (PUVA, 320 nm) to the diseased part producing photosensitization.

Photodynamic therapy (also known as “PDT”) involves the administration of a drug followed by light exposure. In PDT, drugs known as porphyrins are administered intravenously into the body to sensitize diseased tissue to visible light. Forms of porphyrin are well known, they include hematoporphyrin derivative (HPD) and porfimer sodium (Photofrin®) and BPD verteporfin.

Thus, in the present invention, it is contemplated that a nucleic acid segment encoding a keptin may be used in combination with photochemotherapy and photodynamic therapy.

6. Antibiotics/Adrenocortical Hormones

The present invention further contemplates the use of antibiotics in combination with a nucleic acid segment encoding a keptin, in the treatment of hyperprolferative inflammatory skin diseases. Antibiotics include, but are not limited to, amikacin, aminoglycosides (e.g., gentamycin), amoxicillin, amphotericin B, ampicillin, antimonials, atovaquone sodium stibogluconate, azithromycin, capreomycin, cefotaxime, cefoxitin, ceftriaxone, chloramphenicol, clarithromycin, clindamycin, clofazimine, cycloserine, dapsone, doxycycline, ethambutol, ethionamide, fluconazole, fluoroquinolones, isoniazid, itraconazole, kanamycin, ketoconazole, minocycline, of loxacin), para-aminosalicylic acid, pentamidine, polymixin definsins, prothionamide, pyrazinamide, pyrimethamine sulfadiazine, quinolones (e.g., ciprofloxacin), rifabutin, rifampin, sparfloxacin, streptomycin, sulfonamides, tetracyclines, thiacetazone, trimethaprim-sulfamethoxazole, viomycin or combinations thereof.

7. Cancer Gene Therapy

The present invention contemplates that tumor suppressors, antisense oncogenes and pro-apoptotic genes which function to inhibit excessive cellular proliferation may be employed with the present invention. These include but are not limited to the tumor suppressors p53. High levels of mutant p53 have been found in many cells transformed by chemical carcinogenesis, ultraviolet radiation, and several viruses.

Other genes that may be employed according to the present invention include Rb, APC, DCC, NF-1, NF-2, WT-1, MEN-I, MEN-II, zacl, p73, VHL, MMAC1/PTEN, DBCCR-1, FCC, p16, rsk-3, p27, p27/p16 fusions, p21/p27 fusions, anti-thrombotic genes (e.g., COX-1, and inhibitors thereof), ras, myc, raf, fos, and jun; and genes involved in angiogenesis (e.g., VEGF, FGF, thrombospondin, BAI-1, GDAIF, or their receptors).

8. Other Therapeutic Agents

The nucleic acid segment of the present invention may be use with other factors that mediated cell proliferation and inflammation which include but are not limited to those cited herein. Hyperproliferative inflammatory skin disorder may be mediated by any number of molecules such as but not limited to the growth factors: IGF-I, keratinocyte growth factor (KGF), transforming growth factor-alpha, basic fibroblast growth factor (bFGF); the cytokines: (TGF-α), tumour necrosis factor-alpha (TNF-α), interleukin-1, -4, -6 and 8 (IL-1, IL-4, IL-6 and IL-8, respectively); or a combination of one or more of the above. Thus, the present invention contemplates the use of these factors or antagonist thereof in providing a more efficacious therapy for hyperproliferative inflammatory skin disorders.

### VIII. EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

Material and Methods

Cell culture. Keratinocytes and dermal fibroblasts were prepared from newborn human foreskins. After incubation of neonatal foreskins in 0.5% dispase (Roche Molecular Biochemicals, Indianapolis, Ind.) for 3 h at 37° C., the epidermal and dermal sheets were carefully harvested. The epidermal sheets were further treated with 0.3% trypsin for 15 min at 37° C., and isolated epidermal cells were cultured in Keratinocyte-SFM media (Gibco BRL, Grand Island, N.Y.). Third or fourth passaged keratinocytes (60-75% confluency) were used in experiments. The separated dermal tissues were cut into small pieces and placed on culture dishes containing Dulbecco's modified Eagle's medium (DMEM) supplemented with 10% fetal bovine serum. Following over night culture, expanded fibroblasts were collected and cultured in fresh medium.

Treatment of keratinocytes. Keratinocytes were seeded in T-75 flask and grown to 70% confluency. Keratinocyte differentiation was induced by shifting from the SFM medium to the DMEM with 10% FCS. At different time points, cells were harvested and analyzed for mRNA expression of keptin gene. Keratinocytes or HaCaT keratinocyte cell lines were treated with human IL-2 (10 ng/ml), IL-4 12.5 ng/ml, IL-6 (25 ng/ml), IL-10 (5 ng/ml), TNFα (25 ng/ml), IFNγ (500 ng/ml), PMA (100 nM), lipopolysaccharide (LPS) (100 ng/ml), or UVB radiation (100 J/m²). Following 16 h culture, total RNA was isolated and examined for gene expression by Northern blotting.

RNA isolation. Skin biopsies immediately after surgical excision from normal adult volunteers or patients with psoriasis were snap-frozen in a liquid nitrogen and stored at −85° C. until needed. Total RNAs were isolated from these frozen tissue samples, primary cultured keratinocytes, or dermal fibroblasts using RNA-STAT 60 (Tel-TestB, Friendswood, Tex.). Some of isolated RNA samples were further purified for poly(A)⁺ RNAs by PolyA Track mRNA Isolation System IV (Promega, Madison, Wis.).

cDNA cloning of keptin gene. Keptin gene was isolated by differential hybridization with total cDNA probes followed by signal sequence (SS)-trap method (Tashiro et al., 1993). A cDNA library used for the SS-trap method was constructed basically as reported by Tashiro et. al. Briefly, 5 μg of poly(A)⁺ RNA isolated from human keratinocytes was converted into first strand cDNA using SuperScript Choice System with 50 ng of random hexamers (Gibco BRL, Grand Island, N.Y.). After dC tailing of the 3′ end of the first strands by terminal deoxynucleotidyl transferase (Gibco BRL), second strand cDNA was synthesized by SuperScript II reverse transcriptase (Gibco BRL) with dG primer consisting of an EcoRI site and 3 repeats of GGGII (where I is inosin), 5′-GCGGCCGCGAATTCCTGACTAACTGA-3(CGGGII)G-3′. After the synthesis, the double stranded cDNA was ligated to SacI adapters, 5′CCGGATCCGAGCTCGATATCAAGCTTGTACCTC-3′, and size-fractionated on 1.6% agarose electrophoresis. The nucleotide fragments migrated in the range of 300-500 bp were purified by gel extraction kit (Quiagen, Valencia, Calif.) and then PCR-amplified with dG and SacI adapator sequences (cycling protocol: 94° C., 30 sec; 49° C., 1 min; and 72° C., 3 min). Following 25 cycles, the PCR products (1.5 ng) were digested with EcoRI and SacI, and ligated to an expression vector, pcDL-SRα-Tac(3′), which encodes an SS-deficient mutant of human interleukin-2 receptor α (IL-2Rα). An aliquot (30%) of the ligated reaction was used to transform DH10B E. coli (Gibco BRL) by electroporation.

The SS-trap cDNA library was screened for cDNAs expressed preferentially or selectively by keratinocytes using differential hybridization. Briefly, ten thousands of independent colonies were transferred onto nylon membranes and sequentially hybridized with total cDNA probes prepared from polyA+ RNA of keratinocytes and with total cDNA probes from dermal fibroblasts. Through this differential hybridization, approximately 1,500 cDNA clones were selected that showed strong hybridization signals with keratinocyte total cDNA probes and little or no signals with dermal fibroblast probes. The selected clones were randomly sorted into 31 pools (49 clones a pool) for the following SS trap screening. Plasmid DNA (0.2 μg) isolated from each pool (all colonies in a group) was transfected into COS-1 cells (1×104⁴) seeded on 8 well chamber slides (NUNC, Naperville, Ill.) using FuGene 6 (Roche Molecular Biochemicals, Indianapolis, Ind.). Following 3 days in culture, the transfectants were assayed for surface expression of the IL-2R by immunocytochemistry: Fixed cells were incubated with 0.5 μg/ml of mouse monoclonal antibody against human IL-2 Rα (R&D Systems Inc., Minneapolis, Minn.). After incubation with biotinylated anti-mouse IgG, the color was developed by avidin-peroxidase and 3-amino-9-ethylcarbazole (AEC) (DAKO, Carpinteria, Calif.), and staining was examined under microscope. Pools that showed the surface expression were sub-divided (7 clones a subpool) and further screened in an above manner. After second screening, cDNA cones in the positive subpools were then clonally examined for the surface expression.

cDNA clones showing the surface expression were determined for their nucleotide sequences and searched for their identities in GenBank and EST databases using Blast search program provided by National Center for Biotechnology Information (NCBI). Nucleotide sequences were determined for the selected cDNA clones that were found in the EST database.

Because cDNA clones isolated by the SS trap method contain only 5′-end sequences, the full-length cDNA clones were isolated using 3′ rapid amplification of cDNA ends (3′ RACE) system (Gibco BRL, Grand Island, N.Y.). Briefly, 50 ng of poly(A)⁺ RNA isolated from primary cultured keratinocytes was reverse-transcribed to cDNA using oligo (dT)-containing primers, and amplified by PCR with each 0.2 μM of the oligo (dT) primer and oligonucleotide specific to the selected clones (cycling protocol: 94° C., 30 sec; 52-68 62° C., 1 min; and 70° C., 3 min). Following 30 cycles of amplification, an aliquot (1 μl) of the product was further PCR-amplified with nested primers. Resulting PCR products were cloned into pCR2.1 vector by using TA-cloning system (Invitrogen, Carlsbad, Calif.). The clones with longest cDNA inserts were selected and their nucleotide sequences determined. The clones were confirmed as full-length cDNAs by the presence of an open-reading frame and a poly A tail. One of the full-length cDNA clones was designated as keptin gene.

Northern blotting and RT-PCR analysis. Northern blotting was performed as described previously. Briefly, poly(A)⁺ RNAs (2 μg) isolated from primary cultured human keratinocyes, dermal fibroblasts, foreskins, and mouse tissues were size-fractionated on a vertical agarose gel, transferred onto a nylon membrane, and hybridized in buffer (5× SSPE, 10× Denhardt's, 2% SDS, and 100 mg/ml sheared salmon sperm DNA) containing 1×10⁶ cpm/ml of ³²P-labeled cDNA for keptin gene or β-actin. After a 16 h-incubation at 42° C., membranes were washed and autoradiographed. For experiments examining tissue distributions of human keptin mRNA, a nylon membrane (Human Multiple Tissue Blots) with an array of mRNA (2 μg) isolated from various human lymphoid and non-lymphoid organs were purchased from Clontech (Palo Alto, Calif.) and hybridized according to manufacturer's recommendations.

Total RNA isolated from cultured cells and mouse epithelial tissues. RNA (100 ng) was reverse-transcribed and then PCR-amplified in one tube (50 μl) containing 0.2 mM dNTP, 1.2 mM MgCl₂, 20 pmol primers, and SUPERSCRIPT II RT/Taq mix (SUPERSCRIPT™ One-Step™ RT-PCR System, GIBCO-BRL) using a protocol for cDNA synthesis (45° C. for 30 min, and then at 94° C. for 2 min). The PCR cycling protocol consisted of incubation at 94° C. for 45 sec for denaturation; 60° C. for mouse keptin or 55° C. for β-actin primers for 45 sec for annealing; 72° C. for 60 sec for extension; and 30 cycles. PCR products (10 μl) were size-fractionated on 1.2% agarose gel electrophoresis and visualized by ethidium bromide staining. The following primers were used to PCR-amplify cDNA for mouse keptin, 5′-CAGCCCAAACCGGACACCAT-3′ and 5′-GGGGAACTGAGGCAAGGAAGATTT-3′; and for mouse β-actin, 5′-GAGCGGGAAATCGTGC GTGACATT-3′ and 5′-GAAGGTAGTTTCGTGGATGCC-3′.

Computer-assisted analysis of nucleotide and amino acid sequences. Amino acid sequences deduced from the nucleotide sequences of the cDNA clones were predicted for their regional hydropathy by using Kyte-Doolittle method in Protean program (DNASTAR Inc., Madison, Wis.), for signal sequences by SignalP V2.0.b2 (web site, www.cbs.dtu.dk/services/SignalP-2.0, Center for Biological Sequence Analysis, Department of Biotechnology, Technical University of Denmark, Lyngby, Denmark).

Construction of plasmid and adenovirus expression vectors. An expression plasmid vector, pKeptin-Fc, was constructed as follows: An entire coding sequence for human keptin gene was PCR-amplified with primers containing EcoRI and XbaI restriction enzyme sites at 5′ and 3′-end, respectively. The resulting PCR fragment was inserted in frame to a coding sequence for a Fc portion of human IgGl using these sites. The nucleotide sequences of human keptin and the Fc portion were confirmed by DNA sequencing.

Recombinant adenovirus vectors were generated according to manufacturer's manuals of AdEasy system (Quantum Biotechnologies, Carlsbad, Calif.) with minor modification. Briefly, a PCR fragment encoding for mature human keptin was introduced into a CMV-driven shuttle vector (pCMV-shuttle) using XhoI and EcoRV restriction sites. After linearization of this shuttle vector DNA with PmeI, 0.5 μg of the DNA was co-transformed into BJ5183 E. coli with 1 μg of pAdEasy-1 that encodes a whole genome of adenovirus type 5 lacking the E1 and E3 regions. Transformed cells were plated on kanamycine-agar plate to suppress the growth of single transformats with pAdEasy-1 carrying a ampicillin-resistant gene. Obtained plasmid clones were screened for recombined plasmid DNA by their total size (more than 33 kb) and by presence of keptin gene. A selected recombined DNA (5 μg) was removed of plasmid sequences by digestion with PacI and then delivered into a permissive cell line, 293A, using 60 μg of Superfect (Quiagene, Valencia, Calif.). Following 5 day-culture, recombinant viral particles were recovered by free-thawing of transfected cells, amplified by further infection of large numbers of 293A cells, and finally purified by double ultracentrifugation through cesium chloride gradients.

Transfection and infection. One day prior to transfection, COS-1 cells were seeded to 90 mm culture dish at a density of 5×10⁶ cells. Next day, cells were incubated for overnight in the presence of expression vector DNA (5 μg) and 15 μl of Fugene (Roche Diagnostics Corp., Indianapolis, Ind.).

Recombinant adenoviruses were used for gene transduction in COS-1 cells as well as keratinocytes or a human keratinocyte line (HaCaT). Recombinant adenovirus was added to culture of these keratinocytes grown at 24-well plate (1×10⁵ cells/well) at multiplication of infection (MOI) of 250 for COS-1 cells and 500 for keratinocytes.

Protein extraction and SDS-PAGE/Western blotting. One day before harvest, the culture medium was replenished with fresh medium. At 3 days after transfection or infection, the supernatant (1 ml) was recovered. Whole cell extracts were prepared from cells by incubation with 100 μl of 0.3% Triton X-100/PBS containing 1 mM PMSF and 2 μg/ml of leupeptin and subsequent centrifugation. Aliquots (5% each for COS-1 cells and 10% each for keratinocytes) from both fractions were applied to SDS-PAGE on Tris-Tricine gels. Following electro-transfer onto Hybond-P membrane (Amersham Pharmacia Biotech, Piscataway, N.J.), the membranes were blocked with 5% Non-fat dried Milk/01% Tween 20 in PBS and incubated with 0.1 mg/ml of purified rabbit IgG rased against His-tagged keptin recombinant proteins. Signals were developed with ECL plus system (Amersham).

Production of His-tagged keptin recombinant protein. A bacterial expression vector, pET-hKeptin, was constructed by insertion of a PCR-amplified coding sequence for human mature keptin in flame to that for N-terminal 10× Histidine in an expression vector, pET-16b (Novagen, Madison, Wis.). BL21(DE3) E. coli were transformed with pET-hKeptin and treated with 1 mM IPTG for 6 h. After harvesting cells, whole proteins were extracted by Urea buffer [8M urea/100 mM Sodium acetate/Tris-HCl pH 8.0] and applied to affinity purification with nickel resins (Quiagene, Valencia, Calif.). His-tagged keptin was eluted from the resins with a buffer [8M urea/100 mM Sodium acetate/0.5M Imidazole pH 4.5], collected fractions containing proteins, and finally refolded by dialysis against 100 mM phosphate buffer (pH 7.7). Typically, 1 L of bacterial culture yields 5.2 mg of His-tagged keptin. His-tagged DHFR was purified as described before.

Immuno-fluorescent staining of human skin. Cryostat sections of human normal skin or psoriatic skin was prepared and incubated with anti-human keptin antibodies (1 μg.ml) at RT for 30 min. After washing, the sections were fluorescently stained with anti-rabbit antibodies conjugated with FITC, and examined under confocal scanning laser microscopy.

In situ hybridization of human skin. In situ hybridization was performed according to manufacturer's manuals. Briefly, cryostat sections of human normal skin was sequentially pre-treated with 0.2N HCl, 0.2% tritonX-100, and proteinase K (10 μg/ml), and incubated with DNase (50 U/ml). As negative control samples, instead of DNase, we added RNase solution to digest cellular RNA. The treated sections were then incubated in the hybridization buffer of mRNAlocator-Hyb Kit (Ambion, Austin, Tex.) containing predetermined optimal concentrations of biotin-UTP-labeled ribo probe (anti-sense stranded RNA) in vitro synthesized from a keptin cDNA. After 24 h incubation at 42° C., hybridized sections were washed with in situ wash solution and incubated with HRP-conjugated streptavidine, followed by color development with AEC plus H₂O₂ (DAKO-LSAB, Carpinteria, Calif.).

Immuno-electronic microscopic analysis. Human skin specimens were cryoprotected with 15% glycerol/phosphate-buffered slaine (PBS) at 4° C. for 30 min and cryofixed by plunging them into liquid propane cooled to −190° C., followed by cryosubstitution with acetone at −80° C. for 120 h. They were then embedded in Lowicryl K11M (Chemische, Frankfurt, Germany) at −60° C. The specimens were polymerized by ultraviolet irradiation at −60° C. for 72 h and at RT for another 72 h. Ultra-thin sections were incubated for 2 h at 37° C. with anti-human keptin antibodies (1:10 dilution). After washing, each section was placed on a drop of 1 nm gold-labeled goat anti-rabbit IgG (Amersham, Piscataway, N.J.) at a dilution of 1: 40 at RT for 2 h, and then washed with distilled water. The sections were finally counterstained with saturated uranyl acetate and lead citrate for 6 and 2 min, respectively.

Proteinase assays. 4.27 mM (100 ng/ml) Papapin (100 μl) was incubated for 15 min at 37C. in the presence (100 μl) of increasing concentrations of His-tagged keptin or His-tagged control protein (His-DHFR). Fluoro-synthetic substrate for papain, Z-Phe-Arg-MCA (Peptide Institute Incorp., Osaka, Japan) was added at a final concentration of 10 μM and incubated for 3 min. at 37C. in a reaction buffer [50 mM Tris-HCl (pH 7.5)/4 mM EDTA/2 mM DTT]. The reaction was stopped by addition of 30% acetic acid solution, and followed by measurement of fluorescence by 650-10S fluorescence spectro-photomer (Perkin-Elmer, Boston, Mass.) with setting of excitation at 370 nM and emission at 467 nM. For chmotrypsin inhibition by His-tagged keptin, activity of chmotrypsin (4.17 nM, 100 ng/ml) was measured by Suc-Ala-Ala-Pro-Phe-MCA as a substrate and performed in a buffer [50 mM Tris-HCL pH 8.0]. Ficin (300 ng/ml) and Cathepsin B (5 ng/ml, 225 nM) was measured using the same substrate and buffer as used for papain proteinase assays. Trypsin (10 μg/ml, 417 nM) activity was determined by Bz-Arg-MCA and 50 mM Tris-HCL pH 8.0.

### Example 2

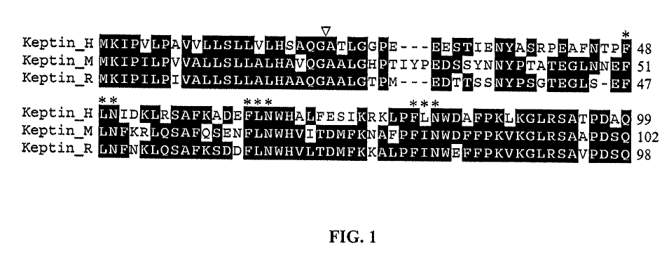

Amino acid sequence of Keptin. From the nucleotide sequence of human keptin gene, the inventors deduced the amino acid sequence. The human keptin precursor as shown in FIG. 1, is cleaved between amino acid 22 and 23 (shown by a reverse traingle). The secreted from of keptin contains three repeats of amino acids FLN as indicated by asterisks.

Epithelial tissue specific expression of keptin mRNA. By Northern Blot analysis, the mRNA expression of keptin in isolated primary cultured cells and various organs from human and mouse were examined. RT-PCR was also used the analysis the expression of RNA isolated from different epithelial tissues of mouse. β-actin was used as an internal control. Keptin was abundantly expressed in human skin as compared to other tissues which demonstrated minimal or no keptin expression. Keratinocytes also showed high expression of keptin as compared to dermal fibroblast cells. Similar results as observed in human tissue were also noted in mouse tissues analyzed. By RT-PCR, keptin was found to be expressed in the skin, oral cavity, tongue, esophagus and stomach in mouse tissue examined. In mouse bladder and uterus keptin expression was minimal. In the large and small intestine expression of keptin was not observed.

Protein expression of human keptin. Human keptin protein was characterized by Western Blotting in the supernatant and whole cell extracts of COS-1 cells or primary cultured keratinocytes transfected with the human keptin gene. COS-1 cells were transfected with an empty vector or with a vector encoding for a fusion protein of an entire sequence of human keptin (including a signal sequence) and a Fc protein of immunoglobulin. At 2 days-post transfection, the supernatant and whole cell extracts were prepared and the small aliquots (10% each) was used for SDS-PAGE analysis followed by western blotting with antibodies raised against human keptin. A single band of 49 kDa was detected in the supernatant but not in the cell extracts. COS-1 cells or keratinocytes were infected with recombinant adenoviruses encoding keptin and LacZ gene at MOI of 250 and 50 respectively. Likewise before, the small aliquots (5% and 10% for COS-1 cells and for keratinocytes, respectively) were examined for expression of keptin protein. Adenoviral keptin was expressed both in the supernatant and the cell extracts of COS-1 cells and keratinocytes with expression of keptin being higher in the cell extracts of either cell type.

Differential distribution of keptin protein in skin tissues. Using frozen skin sections prepared from a normal healthy adult and a psoriatic patient were separately immunostained with anti-keptin antibody. Distribution of keptin proteins was observed under UV flourescent microscopy. Keptin protein distribution was observed throughout the skin of the psoriasis patient but only in the epidermis of the normal healthy adult.

Accumulation of keptin protein in small vesicles of keratinocytes. Intracellular sublocalization of keptin protein was assessed by immunogold electron microscopy using cryoultrathin sections of normal human skin. Immuno-electron microscopic (EM) analysis of human skin was conducted to determine the ultrastructural localization of keptin in granular keratinocytes. Keptin proteins were found inside lamellar granules, known to contain lipids (e.g., fatty acids, cholesterol and its esters, and other ceremide) and enzymes (e.g., sphingomyelinase, acid phosphate, and glycosidase) required for making the lipid envelop, a outer membrane of the cornified cell envelop. Keptin was also present within the granules fused to the apical plasma membrane and those just beneath the membrane. Most interestingly, keptin proteins were released from the membrane-fused granules into the intercellular space. These findings affirms keptin as a secreted protein and that it may be incorporated into the lipid envelop (also co-localized with barrier lipid components and lipid metabolic enzymes). It is important to note that keptin's localization differs from those of elafin, cystatins, SLPI, and PAI-2, which are secretory proteinase inhibitors incorporated into the protein envelop, an inner membrane of the cornified cell envelop.

Expression of keptin mRNA restricted to differential keratinocytes. Primary cultured human keratinocytes were maintained in low-calcium medium and induced for differentiation by shifting to high-calcium medium supplemented with 10% FCS. At the indicated time points, total RNA isolated from treated cells was examined by Northern Blotting for expression of keptin or involucrin mRNA. Keptin expression increased at day 1 and day 2 after exposure to calcium/FCS medium, but decreased by day 4 to minimal levels as in the control. Involucrin epxression remained the same over time. Amounts of ribosomal RNA (rRNA) were similar in all four RNA samples. Normal human skin was in situ hybridized with RNA probes specific to keptin gene. Hybridized signals were detected throughout suprabasal but not in the basal keratinocytes (undifferentiated cell type).

Regulation of keptin gene expression following stimulation. At 15 hours after treatment of keratinocytes with different agents, such as IL-2, IL-4, IL-6, IL-10, TNF alpha, IFN gamma, LPS, PMA and UVB total RNA was isolated and assayed for mRNA expression of the keptin gene. Keratinocytes treated with IL-2, IL-4, IL-6, IL-10, TNF alpha, IFN gamma, and PMA showed expression of keptin. LPS and UVB treated keratinocytes showed minimal expression of keptin. Similar amounts of rRNA showed equal amounts RNA in all lanes of RNA gels.

Analysis of His-tagged keptin recombinant protein. Crude proteins were extracted from E. Coli transformed with a bacterial expression vector encoding a secreted form of keptin tagged with 10× histidine residues at its N-terminus. The extracts were affinity purified with nickel resins and subjected to SDS-PAGE analysis under denatured conditions. Keptin expression was observed in purified cell extract under denatured conditions as compared to crude extracts. The purified protein was also analyzed for its biochemical properties by native gel electrophoresis. Both keptin dimers and monomers were detected in the native gel. Leptin (MW: 14 kDa) was used as a reference for a monomer protein with similar molecular weight to His-Keptin (11.4 kDa).

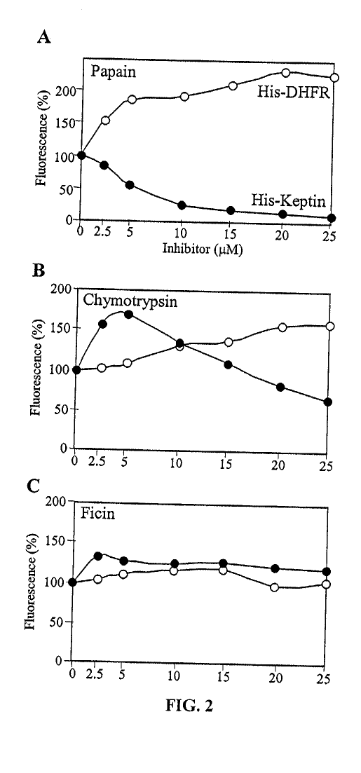

Anti-proteinase activity of His-keptin. Inhibitory ability of His-keptin protein to proteinases was assessed by titration of papain (FIG. 2A), chymotrypsin (FIG. 2B), and ficin (FIG. 2C) at increasing concentrations of 0-25 μM of each inhibitor. His-tagged control protein (His-DFHR) was used as a negative control for all three proteinase assays.

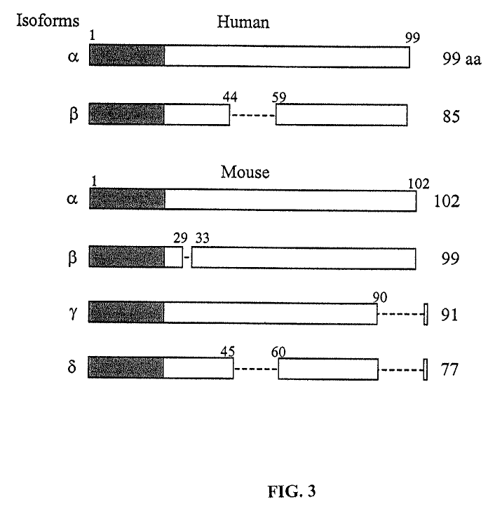

Isoforms of keptin. EST clones containing shorter open reading frames of human and mouse keptin were considered to represent an alternatively spliced mRNA. Amino acid structures were predicted from the nucloetide sequence of the clones, and their products (termed isoform β, γ, and δ) are aligned with a full-length form (isoform α). Amino acid sequences lacking in the truncated isoforms are indicated by dashed lines (FIG. 3).

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

References

The following references, to the extent that they provide exemplary procedural or other details supplementary to those set forth herein, are specifically incorporated herein by reference.

U.S. Pat. No. 4,196,265

U.S. Pat. No. 4,683,202

U.S. Pat. No. 5,302,523

U.S. Pat. No. 5,322,783

U.S. Pat. No. 5,384,253

U.S. Pat. No. 5,399,363

U.S. Pat. No. 5,464,765

U.S. Pat. No. 5,466,468

U.S. Pat. No. 5,538,877

U.S. Pat. No. 5,538,880

U.S. Pat. No. 5,543,158

U.S. Pat. No. 5,550,318

U.S. Pat. No. 5,563,055

U.S. Pat. No. 5,580,859

U.S. Pat. No. 5,589,466

U.S. Pat. No. 5,610,042

U.S. Pat. No. 5,641,515

U.S. Pat. No. 5,656,610

U.S. Pat. No. 5,702,932

U.S. Pat. No. 5,736,524

U.S. Pat. No. 5,780,448

U.S. Pat. No. 5,789,215

U.S. Pat. No. 5,846,225

U.S. Pat. No. 5,846,233

U.S. Pat. No. 5,928,906

U.S. Pat. No. 5,945,100

U.S. Pat. No. 5,981,274

U.S. Pat. No. 5,994,624

Ausubel, Brent, Kingston, Moore, Seidman, Smith, Struhl, In: Current Protocols in Molecular Biology, John, Wiley & Sons, Inc, New York, 1994.

Baichwal and Sugden, In: Gene Transfer, Kucherlapati (ed.), New York, Plenum Press, 117-148, 1986.

Barany and Merrifield, In: The Peptides, Gross and Meienhofer, eds., Academic Press, New York, pp. 1-284 1979.

Chen and Okayama, Mol. Cell Biol., 7(8):2745-2752, 1987.

Coupar et al., Gene, 68:1-10, 1988.

Dykes et al., Brit. J. Dermatol., 122: 173-180; 1990.

Fechheimer et al., Proc. Nat'l Acad. Sci. USA, 84:8463-8467, 1987.

Fraley et al, Proc. Nat'l Acad. Sci. USA, 76:3348-3352, 1979.

Friedmann, Science, 244:1275-1281, 1989.

Gopal, Mol. Cell Biol., 5:1188-1190, 1985.

Graham and Van Der Eb, Virology, 52:456-467, 1973.

Harland and Weintraub, J. Cell Biol., 101:1094-1099, 1985.

Harlow and Lane, Antibodies: A Laboratory manual, Cold Spring Harbor Laboratory, 1988.

Hermonat and Muzycska, Proc. Nat'l Acad. Sci. USA, 81:6466-6470, 1984.

Horwich et al., Virol., 64:642-650, 1990.

Johnson et al., In: Biotechnology And Pharmacy, Pezzuto et al., eds., Chapman and Hall, New York, 1993.

Kaeppler et al., Plant Cell Reports 9: 415-418, 1990.

Kaneda et al, Science, 243:375-378, 1989.

Kato et al, J. Biol. Chem., 266:3361-3364, 1991.

Kyte and Doolittle, i J. Mol. Biol, 157(1):105-32, 1982.

Maniatis, et al., Molecular Cloning, A Laboratory Manual, Cold Spring Harbor Press, Cold Spring Harbor, N.Y., 1990.

Merrifield, Science, 232: 341-347, 1986.

Nicolau and Sene, Biochim. Biophys. Acta, 721:185-190, 1982.

Nicolau et al., Methods Enzymol., 149:157-176, 1987.

PCT App. No. WO 94/09699

PCT App. No. WO 95/06128

Potrykus et al., Mol. Gen. Genet., 199:183-188, 1985.

Potter et al., Proc. Nat'l Acad. Sci. USA, 81:7161-7165, 1984.

Remington's Pharmaceutical Sciences, 15^{th} ed., pages 1035-1038 and 1570-1580, Mack Publishing Company, Easton, Pa., 1980.

Ridgeway, In: Vectors: A survey of molecular cloning vectors and their uses. Rodriguez and Denhardt, eds. Stoneham: Butterworth, pp. 467-492, 1988.

Rippe et al., Mol. Cell Biol., 10:689-695, 1990.

Sambrook et al., Molecular Cloning: A Laboratory Manual, 3rd Ed., Cold Spring Harbor Press, Cold Spring Harbor, N.Y., 2001.

Sambrook et al., Molecular Cloning: A Laboratory Manual, 3rd Ed., Cold Spring Harbor Press, Cold Spring Harbor, N.Y., 2000.

Stewart and Young, Solid Phase Peptide Synthesis, 2d. ed., Pierce Chemical Co. 1984.

Tam et al., J. Am. Chem. Soc., 105:6442, 1983.

Tashiro et al., Science, 261(5121): 600-3, 1993.

Tur-Kaspa et al., Mol. Cell Biol., 6:716-718, 1986.

Wong et al., Gene, 10:87-94, 1980.

Wu and Wu, J. Biol. Chem., 262:4429-4432, 1987.

Wu and Wu, Biochemistry, 27:887-892, 1988.

What is claimed is:

1. An isolated nucleic acid segment encoding a keptin.

2. The isolated nucleic acid segment of claim 1, wherein the keptin is human α keptin, human β keptin, mouse α keptin, mouse γ keptin, mouse γ keptin, mouse δ keptin or rat keptin.

3. The isolated nucleic acid segment of claim 2, wherein the amino acid sequence of the keptin is selected from the group consisting of SEQ. ID. NO: 2, SEQ. ID. NO: 4, SEQ. ID. NO: 6, SEQ. ID. NO: 8, SEQ. ID. NO: 10, SEQ. ID. NO: 12 and SEQ ID NO: 14.

4. The isolated nucleic acid segment of claim 3, wherein the nucleotide sequence of the keptin is selected from the group consisting of SEQ. ID. NO: 1, SEQ. ID. NO: 3, SEQ. ID. NO: 5, SEQ. ID. NO: 7, SEQ. ID. NO: 9, SEQ. ID. NO: 11 and SEQ ID NO: 13.

5. The isolated nucleic acid segment of claim 1, further comprising a promoter.

6. The isolated nucleic acid segment of claim 5, wherein the promoter is a constitutive promoter, an inducible promoter, or a tissue specific promoter.

7. The isolated nucleic acid segment of claim 1, wherein the segment is comprised within an expression vector.

8. The isolated nucleic acid segment of claim 7, wherein the expression vector is a viral vector.

9. The isolated nucleic acid segment of claim 8, wherein the vector is an adenoviral vector, a herpesviral, adeno-associated viral vector, a vaccinia viral vector, a polyoma viral vector or a retroviral vector.

10. The isolated nucleic acid segment of claim 7, further comprising one or more of a polyadenylation signal, an enhancer, an origin of replication, and a selectable marker gene.

11. The isolated nucleic acid segment of claim 7, wherein the expression vector is a non-viral vector.

12. The isolated nucleic acid segment of claim 11, wherein the non-viral vector is comprised within a lipid membrane.

13. A method of expressing a keptin in a cell comprising introducing into the cell an expression vector comprising a nucleic acid segment encoding the keptin and a promoter under conditions that support expression of the keptin.

14. The method of claim 13, wherein the cell is a keratinocyte or a squamous cell carcinoma cell.

15. The method of claim 13, wherein the expression vector is a viral vector or a non-viral vector.

16. The method of claim 13, wherein the keptin is human α keptin, human β keptin, mouse α keptin, mouse β keptin, mouse γ keptin, mouse δ keptin or rat keptin.

17. A method of inhibiting an inflammatory response in a subject comprising providing to the subject an effective amount of a keptin, wherein the keptin inhibits protease activity in keratinocytes.

18. The method of claim 17, wherein the keptin is human α keptin, human β keptin, mouse α keptin, mouse β keptin, mouse γ keptin or mouse δ keptin.

19. The method of claim 17, comprising administering the keptin polypeptide to the subject.

20. The method of claim 17, comprising administering an expression construct encoding the keptin polypeptide to the subject.

21. The method of claim 19, wherein keptin is administered percutaneously or subcutaneously.

22. The method of claim 17, wherein the inflammatory response is associated with a disease condition.

23. The method of claim 22, wherein the disease condition is psoriasis or squamous cell carcinoma.

24. The method of claim 17, further comprising administering to said subject an anti-inflammatory drug.

25. The method of claim 24, wherein the anti-inflammatory drug is selected from the group consisting of a COX inhibitor, a steroid, FK506 (tacrolimus), cyclosporin, and ASM (ascomycin).

26. A method of inhibiting microbial infection in a subject comprising providing to the subject an effective amount of a keptin, wherein the keptin inhibits protease activity in keratinocytes.

27. The method of claim 26, wherein the keptin is human α keptin, human β keptin, mouse α keptin, mouse β keptin, mouse γ keptin or mouse δ keptin.

28. The method of claim 26, comprising administering the keptin polypeptide to the subject.

29. The method of claim 26, comprising administering an expression construct encoding the keptin polypeptide to the subject.

30. The method of claim 28, wherein keptin is administered percutaneously or subcutaneously.

31. The method of claim 26, further comprising administering to said subject an anti-infectious agent.

32. The method of claim 31, wherein the anti-infectious agent is selected from the group consisting of a proteinase inhibitor, an anti-fungal agent, an anti-bacterial agent, an anti-viral agent, and an antibiotic agent.

33. A monoclonal antibody that binds immunologically to a keptin.

34. A hybridoma cell that produces a monoclonal antibody that binds immunologically to a keptin.

35. A polyclonal antibody preparation, antibodies of which bind immunologically to a keptin.

36. An oligonucleotide of 10 to 50 bases in length, wherein said oligonucleotide comprises at least 10 consecutive bases from SEQ ID NO: 1, SEQ. ID. NO: 3, SEQ. ID. NO: 5, SEQ. ID. NO: 7, SEQ. ID. NO: 9, SEQ. ID. NO: 11 or SEQ ID NO: 13.

37. The oligonucleotide of claim 36, wherein the oligonucleotide is 10, 15, 20, 25, 30, 35, 40, 45 or 50 bases in length.

38. The oligonucleotide of claim 36, wherein the number of consecutive bases from SEQ ID NO: 1, SEQ. ID. NO: 3, SEQ. ID. NO: 5, SEQ. ID. NO: 7, SEQ. ID. NO: 9, SEQ. ID. NO: 11 or SEQ ID NO: 13. 10, 15, 20, 25, 30, 35, 40, 45 or 50.

39. A peptide of 10 to 50 residues in length, wherein said peptide comprises at least 10 consecutive residues from SEQ ID NO: 2, SEQ. ID. NO: 4, SEQ. ID. NO: 6, SEQ. ID. NO: 8, SEQ. ID. NO: 10, SEQ. ID. NO: 12 or SEQ ID NO: 14.

40. The peptide of claim 39, wherein the peptide is 10, 15, 20, 25, 30, 35, 40, 45 or 50 residues in length.

41. The peptide of claim 39, wherein the number of consecutive residues from SEQ ID NO: 2, SEQ. ID. NO: 4, SEQ. ID. NO: 6, SEQ. ID. NO: 8, SEQ. ID. NO: 10, SEQ. ID. NO: 12 or SEQ ID NO: 14 is 10, 15, 20, 25, 30, 35, 40, 45 or 50.

42. A method of inhibiting a proteinase comprising contacting to a subject an effective amount of a keptin, wherein the keptin inhibits protease activity in keratinocytes.

43. The method of claim 42, wherein the proteinase is a serine proteinase or a cysteine proteinase.

44. The method of claim 42, wherein said proteinase is located in a cell.

45. The method of claim 42, wherein said proteinase is in a cell-free environment.

46. A pharmaceutical composition comprising a keptin and a pharmaceutically acceptable buffer, diluent or excipient.

47. A pharmaceutical composition comprising an expression construct encoding a keptin and a pharmaceutically acceptable buffer, diluent or excipient.
